(19) United States
(12) Patent Application Publication
(10) Pub. No.: US 2007/0042396 A1
(43) Pub. Date: Feb. 22, 2007

(54) Nucleic acid purification apparatus including photovoltaic device, microfluidic apparatus including the nucleic acid purification apparatus, and method of purifying nucleic acid using the nucleic acid purification apparatus

(76) Inventors: Jong-myeon Park, Seoul (KR); Sang-hyun Peak, Seoul (KR)

Correspondence Address: CANTOR COLBURN, LLP, 55 GRIFFIN ROAD SOUTH, BLOOMFIELD, CT, 06002, US

(21) Appl. No.: 11/347,407

(22) Filed: Feb. 3, 2006

(30) Foreign Application Priority Data
Feb. 3, 2005 (KR) 10-2005-0010184

(51) Int. Cl. C12Q 1/68; C12M 1/34

(52) U.S. Cl. 435/6; 435/287.2; 204/450

(57) ABSTRACT

Provided are a nucleic acid purification apparatus including a chamber that includes an inlet and an outlet, at least two electrodes that defines the chamber and form an electric field, a photovoltaic device that applies a bias to the electrodes, a microfluidic apparatus including the same, and a method of purifying a nucleic acid using the nucleic acid purification apparatus. The nucleic acid purification apparatus includes the photovoltaic device and the conductive transparent electrodes, thus operating independently, and a PCR buffer can be used as an eluate for the nucleic acid. As a result, the nucleic acid purification apparatus can be integrated, and by using the apparatus, a fast and simplified purification can be obtained. The microfludic apparatus including the nucleic acid purification apparatus can operate independently, and perform the purification of the nucleic acid and a PCR at the same time. As a result, the microfluidic apparatus can be miniaturized and automated.

### CROSS-REFERENCE TO RELATED PATENT APPLICATIONS

This application claims the benefit of Korean Patent Application No. 10-2005-0010184, filed on Feb. 3, 2005, in the Korean Intellectual Property Office, the disclosure of which is incorporated herein in its entirety by reference.

### BACKGROUND OF THE INVENTION

1. Field of the Invention

The present invention relates to a nucleic acid purification apparatus including a photovoltaic device and a conductive transparent electrode, and a method of purifying a nucleic acid using the same, and more particularly, to a nucleic acid purification apparatus that includes a photovoltaic device and a conductive transparent electrode so that the nucleic acid purification apparatus can operate independently and can perform the eluting of a separated nucleic acid and a polymerase chain reaction (PCR) at the same time, and a method of purifying a nucleic acid using the same.

2. Description of the Related Art

When a nucleic acid contained in a bimolecular sample is analyzed in a biochip, such as a lab-on-a-chip, to diagnose various diseases, many problems occur. For example, the concentration of a nucleic acid to be analyzed in a sample may be very low. In order to overcome this problem, generally, a nucleic acid is selectively separated, concentrated, and then amplified using a polymerase chain reaction (PCR) technique or the like. Accordingly, the importance of the selective separating and the concentrating before the amplification is increased.

A nucleic acid can be selectively purified using conventional methods.

For example, a material that can selectively bind a nucleic acid can be used. The material may be silica, a glass fiber, an anionic exchange resin, a magnetic bead, or the like.

U.S. Pat. No. 5,234,809 discloses a method of purifying a nucleic acid using a mixture of a starting material, a chaotropic material, and a nucleic acid binding solid material. However, this method requires a lot of time, which is not desirable.

U.S. Pat. No. 6,291,166 discloses a method of purifying a nucleic acid using a solid matrix composed of an alumina with a positive charge. However, it is difficult to perform subsequent analysis because the purified nucleic acid is not separated.

Alternatively, the acid contained in a sample can be purified by an electric field.

For example, WO97/041219 discloses a method of purifying a nucleic acid by an electric field generated by an electrode. Since nucleic acids are negative in general, a negative nucleic acid can be migrated to a positive-biased electrode. However, this method requires an operation of eluting and an operating power source for the generation of the electric field, and thus is not suitable for a small, lightweight, and simplified lab-on-a-chip.

In addition, U.S. Pat. No. 6,518,022 discloses a method of enhancing the hybridization efficiency by forming a probe that can bind a specific nucleic acid on the surface of an electrode when an electric field is applied. That is, a material that can easily bind a nucleic acid is affixed to the surface of the electrode so that a binding yield is increased and the nucleic acid can be selectively purified. However, the method requires modification of the surface of the electrode, which complicates the manufacturing process. In addition, in order to elute the bound nucleic acid, the bound nucleic acid must be eluted in a separated eluting process.

When conventional nucleic acid separation materials are used in conventional nucleic acid purification techniques, purification efficiency is low or a lot of time is required. On the other hand, when an electric field is used, purification time can be decreased, but an operating power source and a separated operation of eluting the separated nucleic acid are required, which is not suitable for a small and lightweight lab-on-a-chip that requires a fast assay.

As a result, an effective nucleic acid purification apparatus that can operate without an external power source and is suitable for a simplified and fast array, thus substantially realizing a lab-on-a-chip, is required.

### SUMMARY OF THE INVENTION

The present invention provides a nucleic acid purification apparatus including a photovoltaic device and a conductive transparent electrode.

The present invention also provides a microfluidic apparatus including the nucleic acid purification apparatus.

The present invention also provides a method of purifying a nucleic acid using the nucleic acid purification apparatus.

According to an aspect of the present invention, there is provided a nucleic acid purification apparatus including: a chamber including an inlet and an outlet; at least two electrodes that define the chamber and generates an electric field; and a photovoltaic device that applies a bias to the electrode.

The electrodes may be formed on a surface of the photovoltaic device.

The electrodes may contact a fluid.

The electrodes may be conductive transparent electrodes.

According to another aspect of the present invention, there is provided a microfluidic apparatus including the above-described nucleic acid purification apparatus.

According to yet another aspect of the present invention, there is provided a method of purifying a nucleic acid, the method including: contacting a sample containing the nucleic acid and at least two conductive transparent electrodes; applying a bias to the conductive transparent electrodes; washing the conductive transparent electrodes; and eluting the nucleic acid using an eluate.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of a nucleic acid purification apparatus according to an embodiment of the present invention;

FIG. 2 is a bar chart illustrating nucleic acid purification efficiency when a nucleic acid labeled with Cy3 is purified by an ITO electrode;

FIG. 3 is an electrophoresis image illustrating polymerase chain reaction (PCR) amplification efficiency after nucleic acid purification; and

FIG. 4 is an electrophoresis image illustrating PCR amplification efficiency in the presence of a PCR inhibitor after nucleic acid purification.

### DETAILED DESCRIPTION OF THE INVENTION

Hereinafter, the present invention will be described in detail by explaining embodiments of the invention with reference to the attached drawings.

Conventional nucleic acid purification apparatuses cannot be used independently because separated outer power sources and nucleic acid eluates are needed, and several operations are required for purification. On the other hand, a nucleic acid purification apparatus according to an embodiment of the present invention includes a photovoltaic device and a conductive transparent electrode so that the nucleic acid purification apparatus can operate independently and a polymerase chain reaction (PCR) buffer can be used as a nucleic acid eluate. As a result, the nucleic acid purification apparatus according to an embodiment of the present invention can be integrated and miniaturized and a fast and simplified purification can be achieved. In addition, by using a microfluidic apparatus including the purification apparatus, the purification of a nucleic acid and PCR can be performed at the same time.

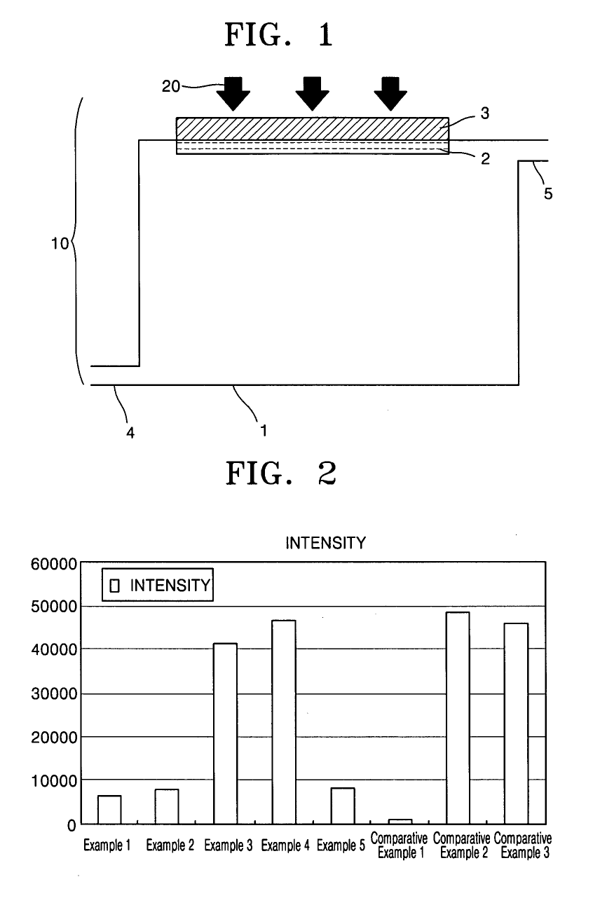

FIG. 1 is a schematic view of a nucleic acid purification apparatus 10 according to an embodiment of the present invention. Referring to FIG. 1, the nucleic acid purification apparatus 10 includes a chamber 1 including an inlet 4 and an outlet 5, an electrode 2 that defines the chamber 1 and generates an electric field in the chamber 1, and a photovoltaic device 3 applying a bias to the electrode 2. A counter electrode connected to the photovoltaic device 3 is omitted in the FIG. 1 for convenience. In the chamber 1 including the inlet 4 and the outlet 5, each of the inlet 4 and the outlet 5 may further include a valve, and the numbers and locations of the inlet 4 and the outlet 5 are not limited to the current embodiment of the present invention. The chamber 1 is not limited to an unpartitioned empty space. That is, in order to facilitate mixing of a fluid and effective purification in the chamber 1, the chamber 1 can be partitioned such that narrow spaces are entirely connected by partial compartments. A plurality of electrodes 2 can be formed in the chamber 1, and in such a case the electrodes 2 can be connected or separated. In addition, any location of an electrode 2 in which the electrode 2 is exposed to the inside of the chamber 1 can be acceptable in the present invention.

The nucleic acid purification apparatus 10 can operate without a separated external power source because the photovoltaic device 3 acts as a power source in the presence of an incident light 20. The photovoltaic device 3 can be easily attached to a surface of the nucleic acid purification apparatus 10 because in general a photovoltaic device is formed in a film. A photovoltaic device formed of silicon exhibits a maximum photovoltaic efficiency of 20% or greater, and can generate voltage of a few volt level so that a voltage required to form an electric field required by the nucleic acid purification apparatus 10 can be supplied. In addition, at such a voltage, current does not flow between electrodes, i.e. electrode 2 and the counter electrode (not shown), and even when a current flows, the current is less than a micro □. Therefore, until the purification is completed, a constnat voltage must be maintained. The incidence light entering the photovoltaic device 3 may be sunlight, but is not limited thereto. That is, any light corresponding to an ultraviolet light region, a visible light region, and an infrared light region can be used in the current embodiment of the present invention.

In the current embodiment of the present invention, the bias applied to the electrode 2 can be controlled according to the distance between electrodes, i.e. electrode 2 and the counter electrode (not shown), and may be in the range of 1 μV to 10 V. Since the only requirement for the electric field is constancy of the field intensity, voltages as small as micro voltages are sufficient to obtain an electric field with a desired intensity even when the distance between electrodes are micrometers. The distance between the electrodes is proportional to the bias. When the bias is less than 1 μm, it is difficult to precisely control the bias. On the other hand, when the bias is greater than 10 V, electrolysis occurs at the surface of the electrode 2.

In addition, when the bias is applied, a smaller current flowing between electrodes is desirable. However, the current flowing between electrodes may be in the range of 1 nA to 1 mA. When the current is greater than 1 mA, electrolysis occurs. Meanwhile, it is difficult to obtain a current of less than 1 nA because a complete insulating state cannot be obtained due to various compounds existing in an electrolyte.

The photovoltaic device 3 is electrically connected to the electrode 2, and the electrode 2 may form a surface of the photovoltaic device 3. When the electrode 2 forms a surface of the photovoltaic device 3, a separated electrode is not required so that the structure of the nucleic acid purification apparatus can be simplified. In addition, since conventional photovoltaic devices include electrodes formed on upper and lower portions thereof, conventional photovoltaic devices can be used in the current embodiment of the present invention. Another electrode may be located opposite to the electrode 2 in the chamber 1, but the location of the opposite electrode is not limited thereto. That is, the opposite electrode can be located in any location only when a uniform electric field is formed in the chamber 1.

The electrode 2 may directly contact a sample containing a nucleic acid. When a positive bias is applied to the electrode 2, a negative nucleic acid migrates in the electric field and is adsorbed to the surface of the electrode 2 which is charged positively. Since it is at the surface of the electrode 2 that the electrostatic force is greatest between the nucleic acid and the electrode 2, the nucleic acid can be effectively adsorbed. As a result, the loss of the nucleic acid in subsequent processes, such as washing or the like, can be minimized.

The electrode 2 can be formed of Au, Pt, Cu, Al, Ti, W, or a metal silicide, preferably, a conductive transparent material. The conductive transparent electrode is mainly used as an electrode in a photovoltaic device and can be interposed between the incident light 20 and the photovoltaic device 3 because a conductive transparent electrode does not block light entering a photovoltaic device.

According to the current embodiment of the present invention, an eluate that is used to elute the nucleic acid adsorbed to the electrode 2 is a PCR buffer. Although a conventional nucleic acid purification technique requires a separated eluate for eluting a nucleic acid adsorbed to an electrode, the nucleic acid purification apparatus 10 according to the current embodiment of the present invention can be used to elute the nucleic acid using only the PCR buffer. In this case, an operation of eluting the nucleic acid by separately injecting an eluate can be omitted and thus the assay time can be reduced and the nucleic acid purification apparatus 10 can be simplified.

In addition, according to an embodiment of the present invention, a microfluidic apparatus including the nucleic acid purification apparatus 10 can perform a PCR when the nucleic acid is adsorbed to the electrode 2. That is, the eluting of the nucleic acid and PCR can occur at the same time, which will be described in detail later when the microfluidic apparatus is described.

The photovoltaic device 3 may be disposed parallel to the horizontal surface of the chamber 1. This structure maximizes the amount of absorbed light because the incident light 20 in general enters perpendicularly to the nucleic acid purification apparatus 10. Since the nucleic acid purification apparatus 10 is formed in a chip shape, more particularly, a wide and thin flat shape, the photovoltaic device 3 can be located on an upper portion or lower portion of the chip-shaped nucleic acid purification apparatus 10. However, the photovoltaic device 3 can be located on another surface of the nucleic acid purification apparatus 10 exposed to the outside, that is, on any surface where the photovoltaic device 3 can be installed.

Although the nucleic acid purification apparatus 10 can be powered by being connected to an outer power source, it is preferred that the nucleic acid purification device 10 can be powered by only the incident light 20 because such an independent operation is a prerequisite for the nucleic acid purification apparatus 10 being an independent apparatus.

In addition, the nucleic acid purification apparatus 10 may further include a controlling device. The controlling device can include a central processing unit requiring a separated power source, preferably a simple circuit that repeats a predetermined sequence over time when a limited power source is considered. In addition, the nucleic acid purification apparatus 10 can be electrically connected to an external apparatus.

The conductive transparent electrode 2 may be formed of indium tin oxide (ITO), fluorine-doped tin oxide (FTO), SnO₂, or the like. Since electrodes formed of these materials are transparent and conductive, they provide the flexibility of the location of the photovoltaic device 3. In addition, the change of temperature resulting from PCR does not affect these electrodes because they can withstand high temperatures.

The photovoltaic device 3 may be silicon type, a compound semiconductor type, or a dye sensitized type. However, the type of the photovoltaic device 3 is not limited thereto. That is, any type of a photovoltaic device that can covert light energy into electrical energy can be used in the current embodiment of the present invention.

The chamber 1 may further include a heat input and output device so that a PCR can occur in the chamber 1. The heat input and output device can be any device that can provide heat to the chamber 1 or absorb heat from the chamber 1, preferably a thermoelectric device.

The microfluidic apparatus including the nucleic acid purification apparatus 10 includes a sample inlet, a sample chamber, a purification and amplification chamber, an assay chamber, and a sample outlet. In detail, the nucleic acid purification apparatus 10 corresponds to the part of comprising the purification and amplification chamber. The sample inlet may have an access from the outside, but when a sample is inserted, the sample inlet can be sealed. The inserted sample, or a cell, is lysised in the sample chamber, and purification of the nucleic acid and the PCR are performed in the purification and amplification chamber. The amplified nucleic acid is sent to the assay chamber and assayed using an analyzing device. The assayed sample is sent to the sample outlet.

The microfluidic apparatus may further include a washing solution, a dissolving reagent, a chamber that stores a PCR mixed solution, a controlling unit that controls a valve connected to the chamber and a pump moving a fluid, and a driving power source.

In the microfluidic apparatus, the elution of the nucleic acid and the PCR of the eluted nucleic acid may occur at the same time. That is, when the nucleic acid is attached to the electrode 2, a PCR buffer used as an eluate is heated. Accordingly, the elution of the nucleic acid and the PCR can occur at the same time. At this time, since the purification of the nucleic acid the PCR can be performed in a single chamber, the structure of the microfluidic apparatus can be simplified. The microfluidic apparatus may be powered by only an incident light. When the microfluidic apparatus is powered only by incident light without a separated external power source, the microfluidic apparatus can be independently used.

The structure of the microfluidic apparatus is not limited to the above-described structure. That is, any structure of the microfluidic apparatus including the nucleic acid purification apparatus 10 known to those skilled in the art can be used.

A method of purifying a nucleic acid according to an embodiment of the present invention will now be described.

First, a sample containing a nucleic acid contacts a conductive transparent electrode. A bias is applied to the electrode so that the nucleic acid is adsorbed to the electrode, and then a washing solution is added to the result to wash the electrode. Then, an eluate is injected and heated so that the nucleic acid is eluted and purified.

According to the current embodiment of the present invention, the eluting of the nucleic acid and aPCR may be performed at the same time. That is, when a PCR buffer is used as the eluate and the temperature of the chamber is changed, the nucleic acid adsorbed to the electrode is eluted and PCR occurs at the same time. Even when a blood that prevents the PCR is present, the PCR can effectively occur.

The PCR buffer, which is used to elute a nucleic acid, may be a material including bovine serum albumin (BSA), but is not limited thereto.

The conductive transparent electrode may be formed of ITO, FTO, SnO₂, or the like.

The bias that is applied to the electrode may be in the range of 2 V to 4 V when electrodes, i.e. electrode 2 and counter electrode (not shown), are separated from each other by a distance of 1 to 1.5 cm. When the bias is less than 1V, it takes a long time for the nucleic acid to move to the electrode. On the other hand, when the bias is greater than 5V, the fluid is subjected to electrolysis. The distance between electrodes is dependent on the voltage applied to the electrode. For example, if the distance between electrodes is small, the required voltage is low.

In detail, the bias applied to the electrode can be adjusted according to the distance between electrodes. The bias may be in the range of 1 μV to 10V. When the bias is applied, a low current is preferable. However, the current may be in the range of 1 nA to 1 mA. When the current exceeds 1 mA, electrolysis occurs. Meanwhile, it is difficult to obtain a current of less than 1 nA because a complete insulating state cannot be obtained due to various chemical compounds contained in an electrolyte.

The present invention will be described in further detail with reference to the following examples and comparative examples. The following examples are for illustrative purposes only and are not intended to limit the scope of the invention.

Purification and Elution of Nucleic Acid Using an Electrode According to the Present Invention

### EXAMPLE 1

2 ml of 50 mM histidine electrolyte containing 1 μM oligonucleotide (31mer; AGG CTT CTT CTT TGC ACC GGT TGG CM ACC A) labeled with Cy3 was provided to an Eppendorf tube. An ITO electrode was used as a working electrode, and a gold-coated wafer was used as an opposite electrode. 5 V was applied between the ITO electrode and the opposite electrode for 5 minutes, and the ITO electrdoe was then washed three times with 5 ml of a distilled water. Then, the ITO electrode was eluted using an eluate. At this time, the eluate was a 10×PCR buffer (100 mM Tris-HCl, 500 mM KCl, 15 mM MgCl₂, Taq polymelase Enzyme 0.5M) containing 0.1 mg/ml of bovine serum albumin (BSA). After the elution, a nucleic acid was PCR amplified by repeating the operation including pre-denaturation (95 □, 1 minute), denaturation (96 □, 5 minutes), annealing (60 □, 13 minutes), and then extension (70□, 15 minutes) 25 times. Then, in order to measure the amount of DNA remaining on the ITO electrode, the relative strength of fluorescence emitting from the fluorescent material was quantitatively measured using a laser scanner (GenePix4000B photon multiplier tube (PMT) 520 obtained from Axon Co.). The results are shown in FIG. 2. FIG. 2 is a bar chart illustrating nucleic acid purification efficiency when a nucleic acid labeled with Cy3 is purified by an ITO electrode

### EXAMPLE 2

An experiment was performed in the same manner as in Example 1 except that the eluate was a 1×PCR buffer containing BSA. The results are shown in FIG. 2.

### EXAMPLE 3

An experiment was performed in the same manner as in Example 1 except that the eluate was a 10×PCR buffer that did not include BSA. The results are shown in FIG. 2.

### EXAMPLE 4

An experiment was performed in the same manner as in Example 1 except that the eluate was a 1×PCR buffer that did not include BSA. The results are shown in FIG. 2.

### EXAMPLE 5

An experiment was performed in the same manner as in Example 1 except that the eluate was 10 mg/ml of BSA instead of the PCR buffer containing BSA. The results are shown in FIG. 2.

### COMPARATIVE EXAMPLE 1

An ITO electrode that did not contact the nucleic acid was used as Comparative Example 1. The results are shown in FIG. 2.

### COMPARATIVE EXAMPLE 2

An experiment was performed in the same manner as in Example 1 except that the eluting of the nucleic acid that was adsorbed to the electrode using the eluate was omitted. The results are shown in FIG. 2.

### COMPARATIVE EXAMPLE 3

An experiment was performed in the same manner as in Example 1 except that the eluate was a hot distilled water. The results are shown in FIG. 2

The ITO electrodes prepared according to Examples 1 through 5 and Comparative Examples 1 through 3 were scanned using a laser and the results are shown in FIG. 2. Referring to FIG. 2, when the ITO according to Comparative Example 1 was laser scanned, the intensity was almost 0 because the nucleic acid was not adsorbed to the ITO electrode. However, the intensity of Comparative Example 3 in which the hot distilled water was used as the eluate was almost equal to the intensity of Comparative Example 2 in which the nucleic acid was not eluted, which indicates that the nucleic acid was not eluted. On the other hand, the intensities of Examples 3 and 4 in which the PCR buffer did not contain BSA were almost equal to the intensity of Comparative Example 3. However, the intensities of Examples 1 and 2 in which the PCR buffer did contain BSA were less than the intensity of Comparative Example 3, which indicates an excellent nucleic acid elution. That is, the ITO layers of Examples 1 and 2 had recovered their original state. The intensity of Example 5 in which only BSA was used was similar to the intensity when the PCR buffer containing BSA was used.

PCR Amplification Efficiency of Nucleic Acid Purified by the Electrode According to the Present Invention

### EXAMPLE 6

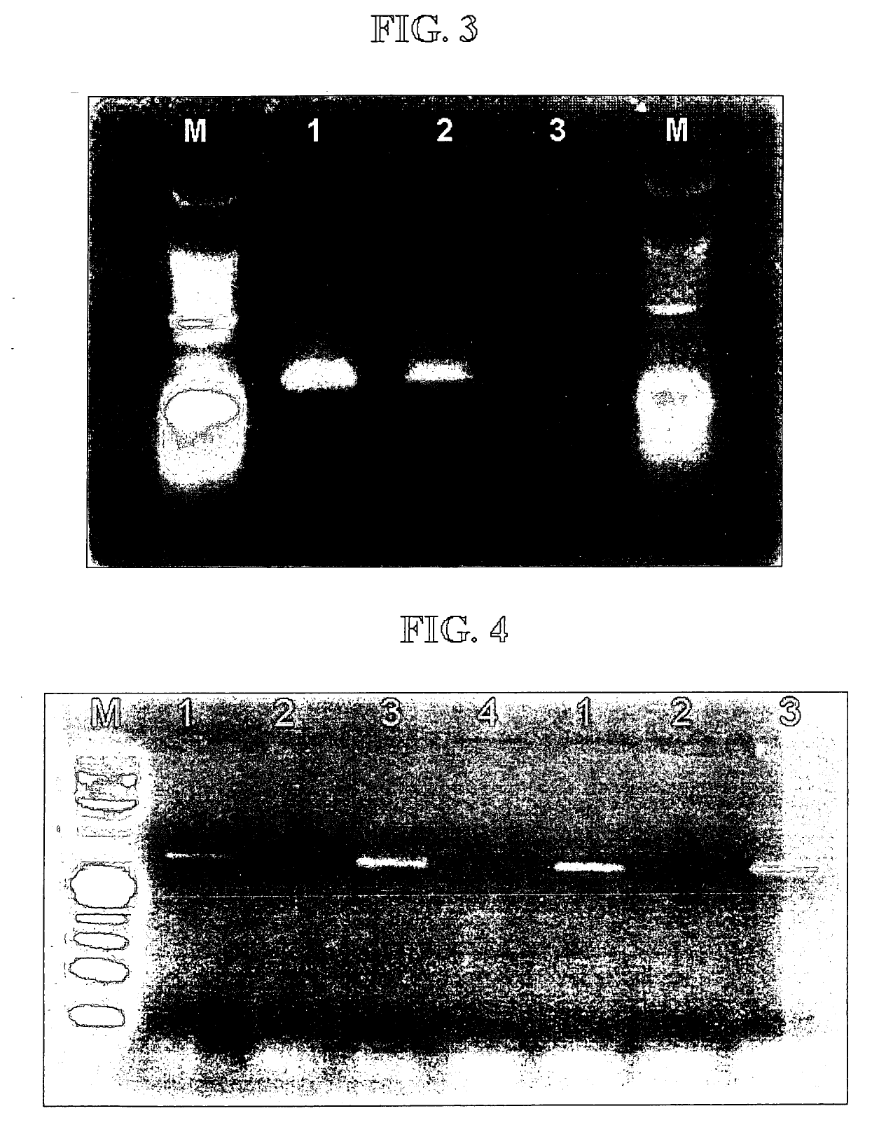

1 ml of 10 ng/μl Staphylococcus Aureus gDNA and 2 ml of a histidine electrolyte containing 50 mM histidine were added to an Eppendorf tube. An ITO electrode was used as a working electrode, and a gold-coated wafer was used as an opposite electrode. 5V was applied between the ITO electrode and the gold-coated wafer for 5 minutes, and then was completely washed with distilled water three times. Then, the ITO electrode was moved to a PCR tube containing a 10×PCR buffer that contained BSA and then a PCR was performed. A first lane shown in FIG. 3 denotes the results. FIG. 3 is an electrophoroesis image illustrating polymerase chain reaction (PCR) amplification efficiency after nucleic acid purification ‘M’ in FIG. 3 denotes a marker.

### COMPARATIVE EXAMPLE 4

1 μl of 10 ng/μl Staphylococcus Aureus gDNA was added to a PCR tube containing a 10×PCR buffer that contained BSA. Then, the ITO having the same size as in Example 6 was inserted to the PCR tube and then a PCR was performed. A second lane shown in FIG. 3 denotes the results.

### COMPARATIVE EXAMPLE 5

An experiment was performed in the same manner as in Example 6 except that a voltage was not applied and the ITO electrode was immersed for 5 minutes. A third lane shown in FIG. 3 denotes the results.

The results of PCR according to Example 6 and Comparative Examples 4 and 5 are shown in FIG. 3. Referring to FIG. 3, the first lane of Example 6 in which the nucleic acid is adsorbed to the electrode by the electric field and the third lane of Comparative Example 5 in which the electric field was not applied were significantly different from each other. In addition, the elution of Example 6 was relatively greater in amount than the elution of Comparative Example 4 in which only gDNA was added.

PCR Amplification Efficiency of Nucleic Acid Purified in the Presence of PCR Inhibitor by the Electrode According to the Present Invention

### EXAMPLE 7

500 μl of 100% blood, 1 μl of 1 ng/μl Klebsiella pneumoniae gDNA, and 2 ml of an histidine electrolyte containing 50 mM of histidine were added to an Eppendorf tube. An ITO electrode was used as a working electrode, and a gold-coated wafer was used as an opposite electrode. 5V was applied between the ITO electrode and the opposite electrode for 5 minutes, and then the electrodes were completely washed with distilled water for three times. Then, the ITO electrode was moved to a PCR tube containing a 10×PCR buffer that contained BSA and then a PCR was performed. The PCR was performed in the same manner as in Example 6. A first lane shown in FIG. 4 denotes the results. FIG. 4 is a photographic/SEM? image illustrating PCR amplification efficiency in the presence of a PCR inhibitor after nucleic acid purification ‘M’ in FIG. 4 denotes a marker.

### EXAMPLE 8

An experiment was performed in the same manner as in Example 7 except that the blood was not added. A third lane shown in FIG. 4 denotes the results.

### COMPARATIVE EXAMPLE 6

The PCR was performed using a 1 μl mixed reacting solution of the 100% blood and gDNA. A second lane shown in FIG. 4 denotes the results.

### COMPARATIVE EXAMPLE 7

An experiment was performed in the same manner as in Example 7 except that the ITO electrode was immersed in the buffer for 5 minutes without the application of the voltage. A fourth lane shown in FIG. 4 denotes the results.

The results of PCR according to Examples 7 and 8 and Comparative Examples 6 and 7 are shown in FIG. 4. Referring to FIG. 4, a band could be obtained from Example 7 in which the nucleic acid was adsorbed using an electric field and Example 8 in which simply the gDNA was added. However, a band could not be obtained from Comparative Example 6 in which a reaction product of the blood and gDNA were used and from Comparative Example 7 in which the electric field is not applied. In general, when a blood is added, no PCR occurs as described in Comparative Example 6 because the blood contains an inhibitor that prevents PCR. However, when the purification was performed using the ITO and the blood is present, as described in Example 7, excellent purification efficiency could be obtained by selectively adsorbing DNA and removing the blood. Accordingly, it was confirmed that a nucleic acid of a biomolecule sample, such as blood or the like, can be effectively purified using the ITO.

A nucleic acid purification apparatus according to the present invention can operate independently by including a photovoltaic device and a conductive transparent electrode. In addition, the nucleic acid purification apparatus uses a PCR buffer as a nucleic acid eluate so that the purification apparatus can be integrated and a fast and simplified purification can be obtained. A microfluidic apparatus including the purification apparatus can operate independently, and the purification of the nucleic acid and a PCR can be performed at the same time. As a result, the microfluidic apparatus can be miniaturized and automated.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A nucleic acid purification apparatus comprising:
a chamber comprising an inlet and an outlet;
at least two electrodes that define the chamber and generate an electric field; and
a photovoltaic device that applies a bias to the electrodes.

2. The nucleic acid purification apparatus of claim 1, wherein the bias applied to the electrodes depends on a distance between the electrodes and is in the range of 1 μV to 10 V.

3. The nucleic acid purification apparatus of claim 2, wherein a current generated when the bias is applied is in the range of 1 nA to 1 mA.

4. The nucleic acid purification apparatus of claim 1, wherein the electrodes are formed on a surface of the photovoltaic device.

5. The nucleic acid purification apparatus of claim 1, wherein the electrodes contact a fluid.

6. The nucleic acid purification apparatus of claim 1, wherein the electrodes are transparent.

7. The nucleic acid purification apparatus of claim 1, wherein the photovoltaic device is parallel to a horizontal surface of the chamber.

8. The nucleic acid purification apparatus of claim 1, powered by only an incident light.

9. The nucleic acid purification apparatus of claim 1, further comprising a controlling device.

10. The nucleic acid purification apparatus of claim 1, electrically connected to an external device.

11. The nucleic acid purification apparatus of claim 1, wherein the electrode is formed of at least one compound selected from a group consisting of indium tin oxide (ITO), fluorine-doped tin oxide (FTO), and SnO₂.

12. The nucleic acid purification apparatus of claim 1, wherein the photovoltaic device is at least one device selected from a group consisting of a silicon type device, a compound semiconductor type device, and a dye sensitized type device.

13. The nucleic acid purification apparatus of claim 1, further comprising a heat input and output device.

14. A microfluidic apparatus comprising the nucleic acid purification apparatus of claim 1.

15. The microfluidic apparatus of claim 14, wherein an elution of the nucleic acid and a polymerase chain reaction (PCR) of the eluted nucleic acid are performed at the same time.

16. The microfluidic apparatus of claim 14, powered by only an incident light.

17. A method of purifying a nucleic acid, the method comprising:
contacting a sample containing the nucleic acid and at least two conductive transparent electrodes;
applying a bias to the conductive transparent electrodes;
washing the conductive transparent electrodes; and
eluting the nucleic acid using an eluate.

18. The method of claim 17, wherein the eluting of the nucleic acid is performed at the same time as when a PCR is performed.

19. The method of claim 17, wherein the eluate is a PCR buffer containing bovine serum albumin (BSA).

20. The method of claim 17, wherein the sample is a blood.

21. The method of claim 17, wherein the conductive transparent electrode is formed of at least one compound selected from a group consisting of indium tin oxide (ITO), fluorine-doped tin oxide (FTO), and SnO₂.

22. The method of claim 17, wherein the bias applied to the conductive transparent electrodes depends on a distance between the electrodes and is in the range of 1 μV to 10 V.

23. The method of claim 22, wherein a current generated when the bias is applied is in the range of 1 nA to 1 mA.
